(19) United States
(12) Patent Application Publication
(10) Pub. No.: US 2012/0244074 A1
(43) Pub. Date: Sep. 27, 2012

(54) LABELLED INTEGRIN BINDERS

(76) Inventor: Magne Solbakken, Oslo (NO)

(21) Appl. No.: 13/510,679

(22) PCT Filed: Dec. 16, 2010
(86) PCT No.: PCT/EP2010/069958

§ 371 Date: May 18, 2012

Related U.S. Application Data
(60) Provisional application No. 61/287,238, filed on Dec. 17, 2009.

(30) Foreign Application Priority Data
Dec. 17, 2009 (GB) 0922014.6

(51) Int. Cl. A61K 51/04; B01L 3/00; C07D 401/14; A61K 49/00; C07D 213/74; C07F 13/00

(52) U.S. Cl. 424/1.89; 546/309; 534/14; 546/277.4; 424/1.65; 424/1.85; 424/9.6; 422/554

(57) ABSTRACT

The present invention provides alternative in vivo imaging agents suitable for use in the detection of αᵥβ₆ expressed in a subject. The invention also provides a method for obtaining said in vivo imaging agents, and use of the in vivo imaging agents in determining αᵥβ₆ expressed in a subject.

### TECHNICAL FIELD OF THE INVENTION

The present invention concerns in vivo imaging and in particular a novel in vivo imaging agent. Also provided by the present invention is a method for the preparation of the in vivo imaging agent of the invention, and a precursor compound useful in said method. The in vivo imaging agent of the invention is useful in the diagnosis of conditions where there is a deviation from normal in the expression of the integrin αᵥβ₆.

### DESCRIPTION OF RELATED ART

The integrins are a family of membrane-bound glycoproteins made up of α and β subunits. The integrin αᵥβ₆ is relatively rare (Busk et al 1992 J Biol Chem; 267(9): 5790); it is formed in epithelial tissue during repair processes and preferentially binds the natural matrix molecules fibronectin (Weinacker et al 1994 J Biol Chem; 269: 6940-6948.) and tenascin (Wang et al, 1996, Am J Respir Cell Mol Biol; 15(5): 664). Vitronectin also binds to αᵥβ₆ (Huang et al 1998 J Cell Sci; 111(15): 2189-2195), and TGF-β is activated by its interaction with αᵥβ₆ (Massagué and Chen 2000 Genes & Dev; 14: 627-644).

The integrin αᵥβ₆ has been found to be upregulated in pathological processes such as wound healing, inflammation and cancer. The ability of αᵥβ₆ to promote migration and invasion has pointed to a potential role as an indicator of cancer aggressiveness (Bates et at J Clin Invest 2005; 115(2): 339-347). Popov et al (J Hepatol 2008; 48: 453-64) have demonstrated that αᵥβ₆ is expressed on activated bile duct epithelia and hepatocytes during the progression of liver fibrosis, thereby suggesting αᵥβ₆ as a target for the treatment of liver fibrosis.

WO 2002/074730 and WO 2005/039547 disclose unlabelled biphenyl compounds that preferentially inhibit the αᵥβ₆ integrin receptor and that can be used as medicaments in a range of disease states, including fibrotic disease states such as liver fibrosis.

The biphenyl compound EMD527040 has been reported as an antagonist of αᵥβ₆ (Popov et al J Hepatol 2008; 48: 453-464). The chemical structure of EMD527040 is as follows:

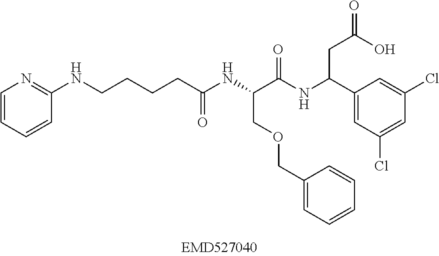

WO 2005/039547 teaches use of EMD527040 in the treatment of conditions in which activated fibroblasts or myofibroblasts are involved. Patsenker et al (Gastroenterology 2008; 135: 660-670) reports that EMD527040 is a specific αᵥβ₆ antagonist. Popov et al (Gastroenterology 2008; 134(4) S1: A-827) refer to a trimer of EMD527040 labelled with ^{99m}Tc for use in the in vivo imaging of liver fibrogenesis. The background liver uptake of the ^{99m}Tc-labelled trimer is reported to be 1.5% of the injected dose, which may be problematic for enabling detection of lesions in or near the liver.

There is scope for alternative in vivo imaging agents that target αᵥβ₆ for use in the diagnosis of a range of disease states associated with abnormal expression of αᵥβ₆.

### SUMMARY OF THE INVENTION

The present invention provides alternative in vivo imaging agents suitable for use in the detection of αᵥβ₆ expressed in a subject. The invention also provides a method for obtaining said in vivo imaging agents, and use of the in vivo imaging agents in determining αᵥβ₆ expressed in a subject. Compared to the prior art, the in vivo imaging agents of the present invention peptides are relatively small, which means they are easier to modify, for example to tailor pharmacokinetic properties. The in vivo imaging agents of the invention also demonstrate similar biological properties to the known αᵥβ₆ antagonist EMD527040.

### DETAILED DESCRIPTION OF THE INVENTION

In one aspect, the present invention provides an in vivo imaging agent of Formula I:

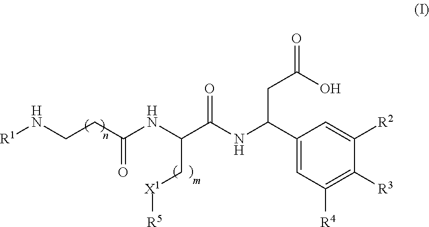

or a salt or solvate thereof, wherein:
n is an integer from 0-6;
m is an integer from 0-8;
X¹ is O, S, or NR′ wherein R′ is hydrogen or C₁₋₄ alkyl;
R¹ is —C(═NH)—NHR′ wherein R′ is as defined for X¹, or R¹ is a C₃₋₅ nitrogen-containing heteroaryl ring having 1 or 2 nitrogen heteroatoms;
each of R²-R⁴ is independently hydrogen, C₁₋₄ alkyl or halogen, or is a substituent comprising an in vivo imaging moiety, or alternatively R² and R³, or R³ and R⁴, together with the carbon atoms to which they are attached, form a C₅₋₆ aryl or C₃₋₅ heteroaryl ring having 1 or 2 heteroatoms;
R⁵ is hydrogen, or is the group R⁶R⁷ wherein R⁶ is a bivalent linker group having 1-50 bivalent linker units selected from an amino acid residue, a carbohydrate residue, —C(OH)—, —(CR′₂)—, —C(═O)—(CR′₂)—, —C(═O)—NR′—, —(CR′₂—O—CR′₂)—, —CR′₂—NR′—, CR′₂—S(O₂)—CR′₂, —(CR′₂)—O—N═CR′—, wherein R′ is as defined for X¹, and wherein R⁷ is hydrogen or is a substituent comprising an in vivo imaging moiety; and,
wherein at least one of R²-R⁴ is a substituent comprising an in vivo imaging moiety, or R⁵ is R⁶R⁷ wherein R⁷ is a substituent comprising an in vivo imaging moiety.

The term “in vivo imaging agent” refers to a chemical compound designed to target a particular physiology or pathophysiology in a subject, and which can be detected following its administration to the subject in vivo.

Suitable salts according to the term “salt or solvate thereof” include (i) physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycolic, gluconic, succinic, methanesulphonic, and para-toluenesulphonic acids; and (ii) physiologically acceptable base salts such as ammonium salts, alkali metal salts (for example those of sodium and potassium), alkaline earth metal salts (for example those of calcium and magnesium), salts with organic bases such as triethanolamine, N-methyl-D-glucamine, piperidine, pyridine, piperazine, and morpholine, and salts with amino acids such as arginine and lysine. Suitable solvates according to the term “salt or solvate thereof” include those formed with ethanol, water, saline, physiological buffer and glycol.

The term “alkyl” means straight-chain or branched-chain alkyl radical containing preferably from 1 to 4 carbon atoms. Examples of such radicals include methyl, ethyl, and propyl.

The term “aryl” refers to a cyclic aromatic radical having 5 to 6 carbon atoms, in the ring system, e.g. phenyl or naphthyl.

A “heteroaryl” substituent is an aryl as defined above wherein at least one of the carbon atoms of the ring has been replaced with a “heteroatom” selected from N, S or O.

The term “nitrogen-containing heteroaryl ring” refers herein to a heteroaryl as defined above wherein the cycle comprises one or two nitrogen heteroatoms.

The term “halogen” encompasses the substituents iodine, bromine, chlorine and fluorine, as well as isotopes thereof suitable for in vivo imaging.

The term “in vivo imaging moiety” refers to an atom or group of atoms that may be detected external to a subject's body following administration to said subject.

The term “substituent comprising an in vivo imaging moiety” refers either to a substituent which is itself an in vivo imaging moiety, or to a chemical group in which is comprised said in vivo imaging moiety. More detail is provided below when specific in vivo imaging moieties are discussed.

The term “amino acid residue” refers to meant a bivalent residue of an L- or a D-amino acid, amino acid analogue (e.g. napthylalanine) or amino acid mimetic which may be naturally occurring or of purely synthetic origin, and may be optically pure, i.e. a single enantiomer and hence chiral, or a mixture of enantiomers. Preferably the amino acids of the present invention are optically pure.

The term “carbohydrate residue” refers to a bivalent aldehyde or a ketone derivative of a polyhydric alcohol. It may be a monomer (monosaccharide), such as fructose or glucose, or two sugars joined together to form a disaccharide. Disaccharides include sugars such as sucrose, which is made of glucose and fructose. The term “sugar” includes both substituted and non-substituted sugars, and derivatives of sugars. Preferably, the sugar is selected from glucose, glucosamine, galactose, galactosamine, mannose, lactose, fucose and derivatives thereof, such as sialic acid, a derivative of glucosamine. The sugar is preferably α or β. The sugar may especially be a manno- or galactose pyranoside. The hydroxyl groups on the sugar may be protected with, for example, one or more acetyl groups. The sugar moiety is preferably N-acetylated. Preferred examples of such sugars include N-acetyl galactosamine, sialic acid, neuraminic acid, N-acetyl galactose, and N-acetyl glucosamine.

Where a particular in vivo imaging agent of the present invention comprises a chiral centre, the scope of the invention also includes compositions comprising the racemic mixture of the two enantiomers, as well as compositions comprising each enantiomer individually substantially free of the other enantiomer. Thus, for example, contemplated herein is a composition comprising the S enantiomer substantially free of the R enantiomer, or a composition comprising the R enantiomer substantially free of the S enantiomer. By “substantially free” it is meant that the composition comprises less than 10%, or less than 8%, or less than 5%, or less than 3%, or less than 1% of the minor enantiomer. If the particular in vivo imaging agent comprises more than one chiral center, the scope of the present disclosure also includes compositions comprising a mixture of the various diastereomers, as well as compositions comprising each diastereomer substantially free of the other diastereomers. The recitation of an in vivo imaging agent, without reference to any of its particular diastereomers, includes compositions comprising all four diastereomers, compositions comprising the racemic mixture of R,R and S,S isomers, compositions comprising the racemic mixture of R,S and S,R isomers, compositions comprising the R,R enantiomer substantially free of the other diastereomers, compositions comprising the S,S enantiomer substantially free of the other diastereomers, compositions comprising the R,S enantiomer substantially free of the other diastereomers, and compositions comprising the S,R enantiomer substantially free of the other diastereomers.

Preferably, n of Formula I is from 1-4, most preferably 3.

Preferably, m of Formula I is from 0-3, most preferably 1.

Preferably, X¹ of Formula I is NR′, most preferably NH.

Preferably, R¹ of Formula I is a 5- or 6-membered nitrogen-containing heteroaryl ring, most preferably pyridyl or imidazolyl. Where R¹ is pyridyl it is preferably 2-pyridyl.

Preferably, each of R²-R⁴ of Formula I is independently hydrogen, chloro, iodo or fluoro, most preferably hydrogen, iodo or chloro.

Preferably, R⁵ of Formula I is the group R⁶R⁷. When X¹ is NH, R⁶R⁷ is preferably the group —C(═O)—R^{6a}R^{7a} wherein R^{6a} is a bivalent linker group having 1-20 bivalent linker units, wherein said bivalent linker units are as defined above for R⁶, and wherein R^{7a} is as defined above for R⁷.

A preferred in vivo imaging moiety for Formula I is selected from:
(i) a radioactive metal ion;
(ii) a paramagnetic metal ion;
(iii) a gamma-emitting radioactive halogen;
(iv) a positron-emitting radioactive non-metal;
(v) a reporter suitable for in vivo optical imaging.

When the imaging moiety is a radioactive metal ion, i.e. a radiometal, suitable radiometals can be either positron emitters such as ⁶⁴Cu, ⁴⁸V, ⁵²Fe, ⁵⁵Co, ^{94m}Tc or ⁶⁸Ga; γ-emitters such as ^{99m}Tc, ¹¹¹In, ^{113m}In, or ⁶⁷Ga. Preferred radiometals are ^{99m}Tc, ⁶⁴Cu, ⁶⁸Ga and ¹¹¹In. Most preferred radiometals are γ-emitters, especially ^{99m}Tc.

When the imaging moiety is a paramagnetic metal ion, suitable such metal ions include: Gd(III), Mn(II), Cu(II), Cr(III), Fe(III), Co(II), Er(II), Ni(II), Eu(III) or Dy(III). Preferred paramagnetic metal ions are Gd(III), Mn(II) and Fe(III), with Gd(III) being especially preferred.

When the imaging moiety is a gamma-emitting radioactive halogen, the radiohalogen is suitably chosen from ¹²³I, ¹³¹I or ⁷⁷Br. ¹²⁵I is specifically excluded as it is not suitable for use as an imaging moiety for diagnostic imaging. A preferred gamma-emitting radioactive halogen is ¹²³I.

When the imaging moiety is a positron-emitting radioactive non-metal, suitable such positron emitters include: ¹¹C, ¹³N, ¹⁵O, ¹⁷F, ¹⁸F, ⁷⁵Br, ⁷⁶Br or ¹²⁴I. Preferred positron-emitting radioactive non-metals are ¹¹C, ¹³N, ¹⁸F and ¹²⁴I, especially ¹¹C and ¹⁸F, most especially ¹⁸F.

When the imaging moiety is a reporter suitable for in vivo optical imaging, the reporter is any moiety capable of detection either directly or indirectly in an optical imaging procedure. The reporter might be a light scatterer (e.g. a coloured or uncoloured particle), a light absorber or a light emitter. More preferably the reporter is a dye such as a chromophore or a fluorescent compound. The dye can be any dye that interacts with light in the electromagnetic spectrum with wavelengths from the ultraviolet light to the near infrared. Most preferably the reporter has fluorescent properties. Preferred organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes. Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots). Particular examples of chromophores which may be used include: fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. Particularly preferred are dyes which have absorption maxima in the visible or near infrared (NIR) region, between 400 nm and 3 μm, particularly between 600 and 1300 nm. Optical imaging modalities and measurement techniques include, but not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarisation, luminescence, fluorescence lifetime, quantum yield, and quenching. A preferred a reporter suitable for in vivo optical imaging is a Cy dye.

A most preferred in vivo imaging moiety is a radioactive metal ion, a gamma-emitting radioactive halogen, a positron-emitting radioactive non-metal, or a reporter suitable for in vivo optical imaging each as suitably and preferably defined above.

Preferably, for the in vivo imaging agent of Formula I, one of R²-R⁴ is ¹⁸F or ¹²³I.

Alternatively preferably, for the in vivo imaging agent of Formula I, R⁵ is the group R⁶R⁷ wherein R⁷ comprises an imaging moiety selected from ¹⁸F, a metal complex comprising either a radioactive metal ion or a paramagnetic metal ion or a reporter suitable for optical imaging. As previously recited herein, when X¹ is NH, R⁶R⁷ is preferably —C(═O)—R^{6a}R^{7a}, and for this embodiment R^{7a} preferably comprises said imaging moiety.

By the term “metal complex” is meant a coordination complex of the metal ion with one or more ligands. It is strongly preferred that the metal complex is “resistant to transchelation”, i.e. does not readily undergo ligand exchange with other potentially competing ligands for the metal coordination sites. Potentially competing ligands may be in the precursor compound itself, or in other excipients in the preparation in vitro (e.g. radioprotectants or antimicrobial preservatives used in the preparation), or endogenous compounds in vivo (e.g. glutathione, transferrin or plasma proteins).

The bivalent linker group of Formula I is preferably a chain of between 10 and 50 atoms, and most preferably a chain of between 10 and 30 atoms. Most preferably, the bivalent linker group acts as a biomodifier moiety. A “biomodifier moiety” has the function of modifying the pharmacokinetics and blood clearance rates of the in vivo imaging agent of Formula I. An example of a suitable biomodifier moiety is one based on a monodisperse PEG building block comprising 1 to 20 units of said building block. Additionally, said biomodifier moiety may also represent 1 to 10 amino acid residues. Preferred amino acid residues for said biomodifier moiety are charged amino acids such as lysine and glutamic acid, or charged non-natural amino acids such as cysteic acid and phosphonoalanine. In addition, the amino acids glycine, aspartic acid and serine may be included.

A preferred in vivo imaging agent of Formula I is of Formula Ia:

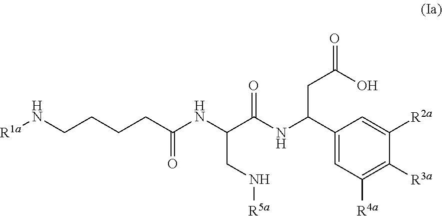

or a salt or solvate thereof, wherein:
R^{1a} is as suitably and preferably defined above for R¹;
R^{2a}-R^{4a} are as suitably and preferably defined above for R²-R⁴;
R^{5a} is hydrogen, or is the group —C(═O)—R^{6a}R^{7a} wherein R^{6a} and R^{7a} are as suitably and preferably defined above.

Non-limiting examples of preferred in vivo imaging agents according to the present invention are as follows:

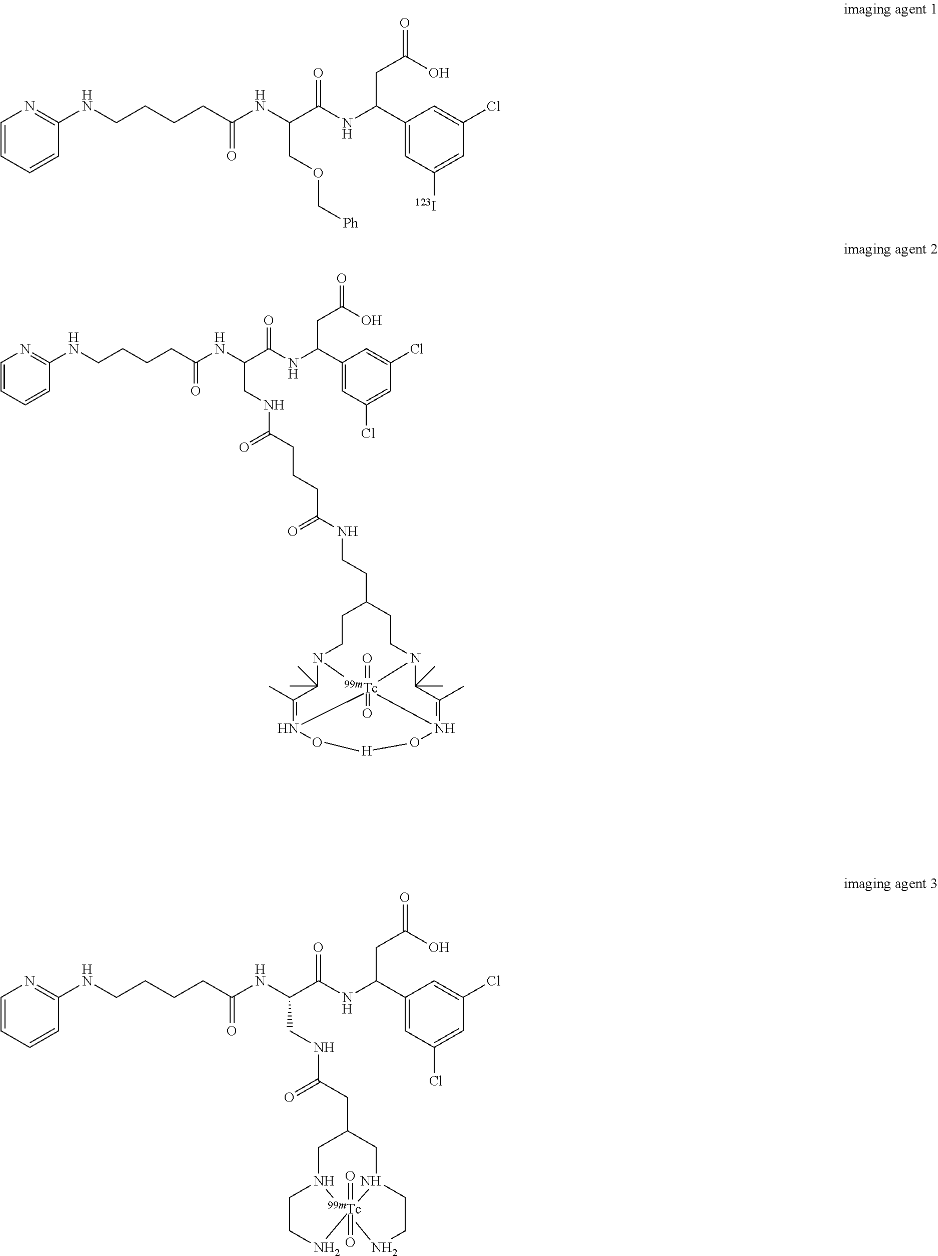

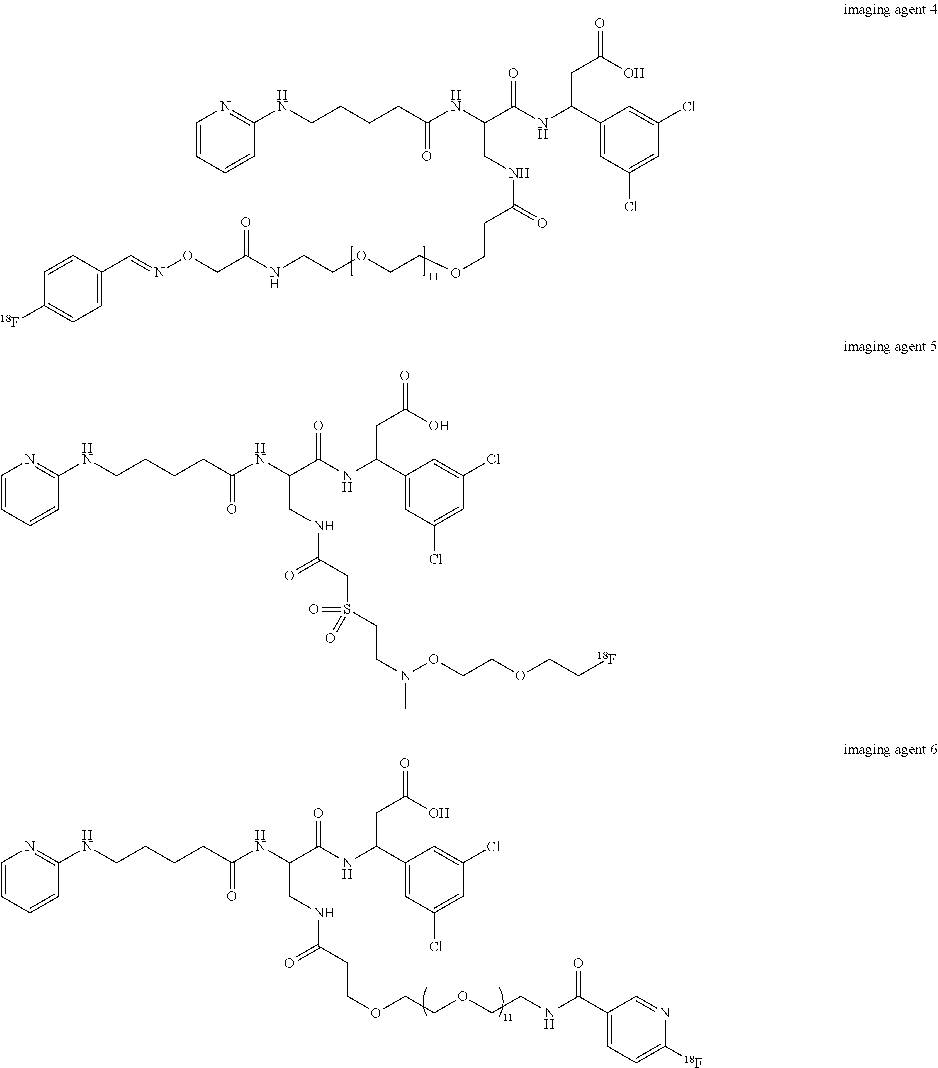

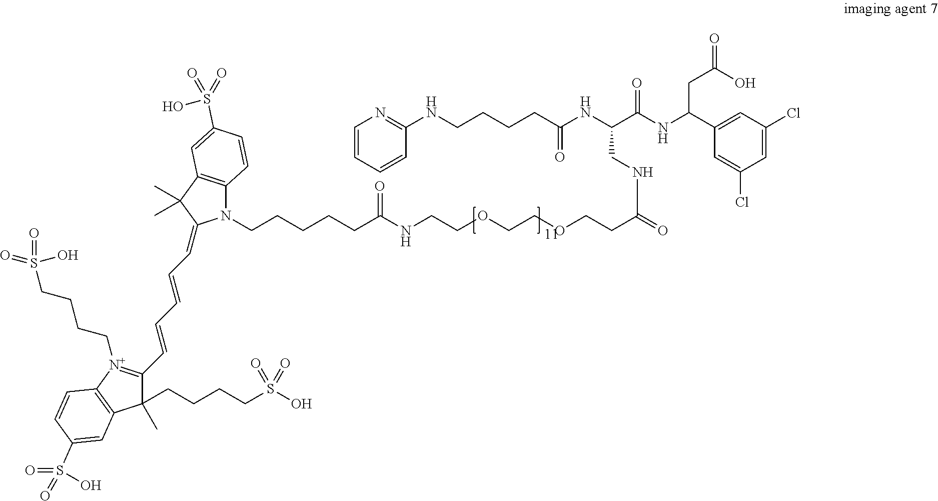

In another aspect, the present invention provides a method for the preparation of the in vivo imaging agent of Formula I as suitably and preferably defined herein, wherein said method comprises reacting a suitable source of an in vivo imaging moiety with a precursor compound of Formula II:

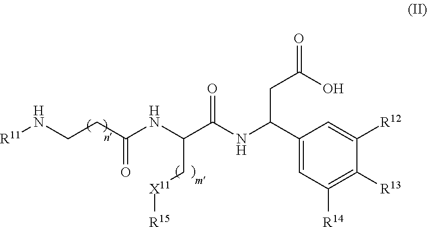

wherein:
R¹¹ is as suitably and preferably defined for R¹ of Formula I;
R¹²-R¹⁴ are independently selected from hydrogen, C₁₋₄ alkyl or halogen, or a precursor group;
R¹⁵ is hydrogen or a precursor group, or is the group R¹⁶R¹⁷ wherein R¹⁶ is a bivalent linker group as suitably and preferably defined herein for R⁶ of Formula I, and wherein R¹⁷ is hydrogen or is a precursor group; and,
X¹¹ is as suitably and preferably defined herein for X¹ of Formula I;
m′ is as suitably and preferably defined herein for m of Formula I;
n′ is as suitably and preferably defined for n of Formula I;
wherein at least one of R¹²-R¹⁵ is a precursor group, or R¹⁵ is R¹⁶R¹⁷ wherein R¹⁷ is a precursor group;
and wherein any reactive groups other than said precursor group are chemically protected.

Broadly speaking, the step of “reacting” the precursor compound with the suitable source of an in vivo imaging moiety involves bringing the two reactants together under reaction conditions suitable for formation of the desired in vivo imaging agent in as high a yield as possible.

A “suitable source of an in vivo imaging moiety” means the in vivo imaging moiety in a chemical form that is reactive with a precursor group in the precursor compound such that the in vivo imaging moiety becomes covalently attached, resulting in the in vivo imaging agent of the invention.

A “precursor compound” comprises a derivative of the in vivo imaging agent, designed so that chemical reaction with a convenient chemical form of an in vivo imaging moiety occurs site-specifically; can be conducted in the minimum number of steps (ideally a single step); and without the need for significant purification (ideally no further purification), to give the desired in vivo imaging agent. Such precursor compounds are synthetic and can conveniently be obtained in good chemical purity. The precursor compound may optionally comprise one or more protecting groups for certain functional groups, or “reactive groups”, in order to avoid unwanted reactions.

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well-known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde (1-(4,4-dimethyl-2,6-dioxocyclohexylidene)ethyl) or Npys (3-nitro-2-pyridine sulfenyl); and for carboxyl groups: methyl ester, tert-butyl ester or benzyl ester. For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tetrabutyldimethylsilyl. For thiol groups, suitable protecting groups are: trityl and 4-methoxybenzyl. A substituent herein comprising a protecting group is “chemically protected”. The use of protecting groups is described in ‘Protective Groups in Organic Synthesis’, Theorodora W. Greene and Peter G. M. Wuts, (Fourth Edition, John Wiley & Sons, 2006).

A “precursor group” is a chemical group that preferentially reacts with the suitable source of an in vivo imaging moiety in order to obtain the in vivo imaging agent.

For the preferred in vivo imaging agents of Formula Ia, said precursor compound of Formula II is of Formula IIa:

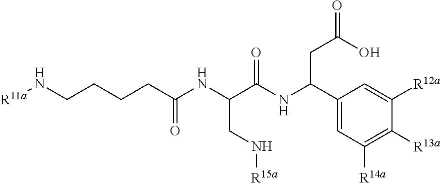

wherein R^{11a}-R^{15a} are as defined above for R¹¹-R¹⁵ of Formula II.

Scheme 1 below is based on the method disclosed by Goodman et al (2002 J Med Chem; 45: 1045-1051), and describes the initial steps in the preparation of a precursor compound of Formula II as suitably and preferably defined above.

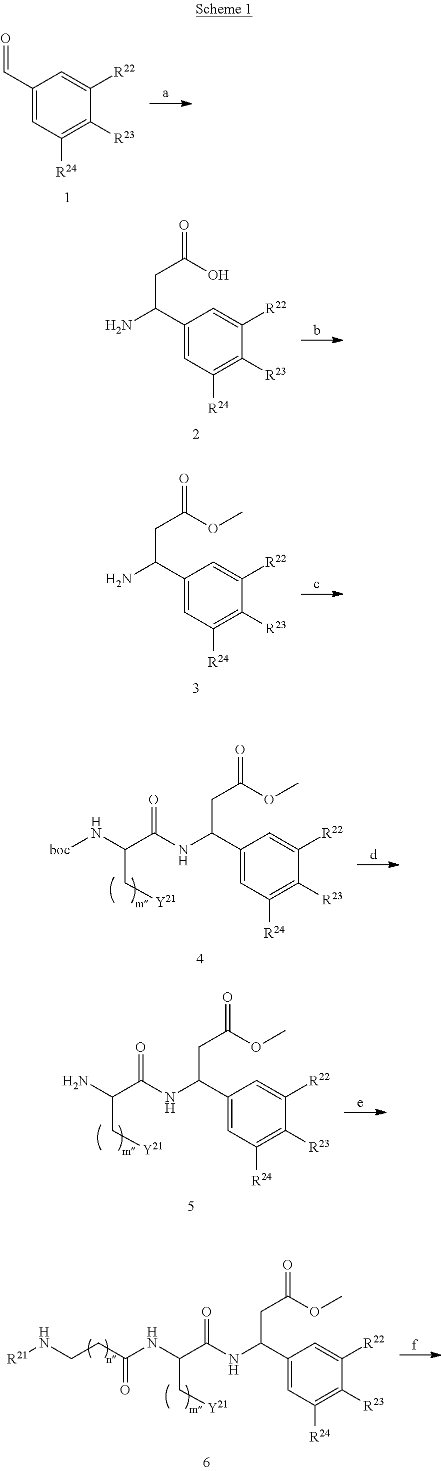

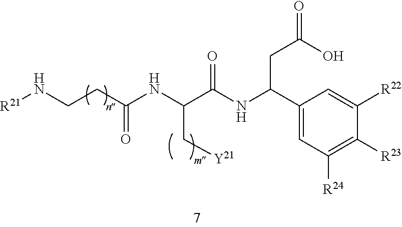

wherein:

R²¹ is as defined above for R¹ of Formula I;

R²²-R²⁴ are independently selected from hydrogen, C₁₋₄ alkyl or halogen, or alternatively R²² and R²³, or R²³ and R²⁴, together with the carbon atoms to which they are attached, form a C₃₋₆ aryl or C₃₋₆ heteroaryl ring having 1 or 2 heteroatoms;

Y²¹ is —X²¹—R²⁵ wherein X²¹ is as defined above for X¹ of Formula I, and R²⁵ is hydrogen or a protecting group;

m″ is as defined for m of Formula I; and,

n″ is as defined for n of Formula I.

In Scheme 1 above, the commercially-available aldehyde 1 is reacted in step (a) with malonic acid and ammonium acetate to obtain intermediate 2. Reaction of intermediate 2 in step (b) with methanol and SOCl₂.results in intermediate 3. Using standard peptide coupling reagents and methods, intermediate 3 is reacted in step (c) with Boc-NH—CH((CH₂)_{m″}—Y²¹)—C(═O)OH (various reagents of this type are available commercially from vendors such as Novabiochem, Bachem, Advanced Chemtech e.g. Boc-Lys(Fmoc)-OH, Boc-Lys(Z)—OH, or corresponding diaminopropionic acids, e.g. Boc-Dpr(Fmoc)-OH). The Boc protecting group is removed in step (d) using standard methods, e.g. 2M HCl in dioxane, to obtain intermediate 5. In step (e) intermediate 5 is reacted with R²¹—NH—(CH₂)ₙ₁—C(═O)—OH, this reactant prepared by following or adapting the method described by Goodman et al (2002 J Med Chem; 45: 1045-1051). The resultant intermediate 6 is reacted in step (f) with NaOH in a suitable solvent to convert the ester into the corresponding carboxylic acid and therefore arrive at intermediate 7. Intermediate 7 can then be modified using methods well-known to the skilled person to introduce precursor groups at any of R²²-R²⁴, or at Y²¹ in order to arrive at the precursor compound of Formula II as defined herein. Such methods are described in more detail below in the discussion of particular in vivo imaging moieties.

Preferably for Formula II one of R¹²-R¹⁴ is said precursor group.

Alternatively preferably for Formula II, R¹⁵ is R¹⁶R¹⁷ wherein R¹⁷ is said precursor group.

A preferred precursor group of Formula II is selected from:
(i) one or more ligands capable of complexing a metallic imaging moiety;
(ii) an organometallic derivative such as a trialkylstannane or a trialkylsilane;
(iii) a derivative containing an alkyl halide, alkyl tosylate or alkyl mesylate for nucleophilic substitution;
(iv) a derivative containing an aromatic ring activated towards nucleophilic or electrophilic substitution;
(v) a derivative containing a functional group susceptible to acylation;
(vi) a derivative containing a functional group that takes part in oxime formation when reacted with a benzaldehyde;
(vii) a derivative containing a vinylsulfone functional group;
(viii) a derivative containing a functional group which undergoes facile alkylation; or,
(ix) a derivative which alkylates thiol-containing compounds to give a thioether-containing product.

Where the precursor group of the precursor compound of Formula II is either (i) or (ii), the precursor compound of Formula II forms another aspect of the present invention.

It is well-known in the art of in vivo imaging agents which precursor group to select for reaction with a particular source of in vivo imaging moiety. This is described hereunder in more detail to guide the reader as to how to obtain particular in vivo imaging agents of the invention.

Where the in vivo imaging moiety is a metal ion, the precursor group comprises one or more ligands capable of complexing a metallic imaging moiety. Suitable ligands for use in the present invention which form metal complexes resistant to transchelation include: chelating agents, where 2-6, preferably 2-4, metal donor atoms are arranged such that 5- or 6-membered chelate rings result (by having a non-coordinating backbone of either carbon atoms or non-coordinating heteroatoms linking the metal donor atoms); or monodentate ligands which comprise donor atoms which bind strongly to the metal ion, such as isonitriles, phosphines or diazenides. Examples of donor atom types which bind well to metals as part of chelating agents are: amines, thiols, amides, oximes, and phosphines. Phosphines form such strong metal complexes that even monodentate or bidentate phosphines form suitable metal complexes. The linear geometry ofisonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as MIBI (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

Suitable chelating agents which form metal complexes resistant to transchelation include, but are not limited to:

(i) diaminedioximes;

(ii) N₃S ligands having a thioltriamide donor set such as MAG₃ (mercaptoacetyltriglycine) and related ligands; or having a diamidepyridinethiol donor set such as Pica;

(iii) N₂S₂ ligands having a diaminedithiol donor set such as BAT or ECD (i.e. ethylcysteinate dimer), or an amideaminedithiol donor set such as MAMA;

(iv) N₄ ligands which are open chain or macrocyclic ligands having a tetramine, amidetriamine or diamidediamine donor set, such as cyclam, monoxocyclam dioxocyclam; and,

(v) N₂O₂ ligands having a diaminediphenol donor set;

The above described ligands are particularly suitable for complexing technetium e.g. ^{94m}Tc or ^{99m}Tc, and are described more fully by Jurisson et al (1999 Chem Rev; 99: 2205-2218). The ligands are also useful for other metals, such as copper (⁶⁴Cu or ⁶⁷Cu), vanadium (e.g. ⁴⁸V), iron (e.g. ⁵²Fe), or cobalt (e.g. ⁵⁵Co). Other suitable ligands are described in WO9101144, including ligands which are particularly suitable for indium, yttrium and gadolinium, especially macrocyclic aminocarboxylate and aminophosphonic acid ligands. Ligands which form non-ionic (i.e. neutral) metal complexes of gadolinium are known and are described in U.S. Pat. No. 4,885,363. Particularly preferred for gadolinium are chelates including DTPA, ethylene diamine tetraacetic acid (EDTA), triethylene tetraamine hexaacetic acid (TTHA), 1,4,7,10-tetraazacyclododecane-1,4,7,10-tetraacetic acid (DOTA), 10-(2-hydroxypropyl)-1,4,7,10-tetraazacyclododecane-1,4,7-triacetic acid (DO3A) and derivatives of these.

Making reference to Scheme 1 above, precursor compounds where the precursor group is a chelating agent can be obtained starting with compound 7 where Y²¹ is —X²¹—R²⁵ where R²⁵ is a protecting group. Step (f) of Scheme 1 can then be followed by removal of the R²⁵ protecting group. A reactive derivative of the chelating agent is reacted with the deprotected compound 7 to obtain the precursor compound. A reactive derivative can for example be an active ester derivative or a carboxylic acid derivative, for reaction with NH₂ in deprotected compound 7. Preferably when the precursor group is a chelating agent, R¹⁵ is the group R¹⁶R¹⁷, wherein R¹⁷ is the chelating agent. In this case, before the addition of the chelating agent, the deprotected compound 7 is reacted with a reactive derivative of the bivalent linker group R¹⁶.

For ^{99m}Tc labelling of a precursor compound of Formula II comprising a suitable chelating agent, the usual technetium starting material is pertechnetate, i.e. TcO₄⁻ which is technetium in the Tc(VII) oxidation state. Pertechnetate itself does not readily form metal complexes, hence the preparation of technetium complexes usually requires the addition of a suitable reducing agent such as stannous ion to facilitate complexation by reducing the oxidation state of the technetium to the lower oxidation states, usually Tc(I) to Tc(V). The solvent may be organic or aqueous, or mixtures thereof. When the solvent comprises an organic solvent, the organic solvent is preferably a biocompatible solvent, such as ethanol or DMSO. Preferably the solvent is aqueous, and is most preferably isotonic saline.

A precursor compound of Formula II suitable for preparing radioiodinated in vivo imaging agents of Formula I comprises a derivative which either undergoes electrophilic or nucleophilic radioiodination or undergoes condensation with a labelled aldehyde or ketone. Examples of the first category are:
(a) organometallic derivatives such as a trialkylstannane (e.g. trimethylstannyl or tributylstannyl), or a trialkylsilane (e.g. trimethylsilyl) or an organoboron compound (e.g. boronate esters or organotrifluoroborates);
(b) a non-radioactive alkyl bromide for halogen exchange or alkyl tosylate, mesylate or triflate for nucleophilic iodination;
(c) aromatic rings activated towards nucleophilic iodination (e.g. aryl iodonium salt aryl diazonium, aryl trialkylammonium salts or nitroaryl derivatives).

A preferred such precursor compound comprises: a non-radioactive halogen atom such as an aryl iodide or bromide (to permit radioiodine exchange); an organometallic precursor compound (e.g. trialkyltin, trialkylsilyl or organoboron compound); or an organic precursor such as triazenes or a good leaving group for nucleophilic substitution such as an iodonium salt. Preferably for radioiodination, the precursor compound comprises an organometallic precursor compound, most preferably trialkyltin.

Precursor compounds and methods of introducing radioiodine into organic molecules are described by Bolton (2002 J Lab Comp Radiopharm: 45: 485-528). Suitable boronate ester organoboron compounds and their preparation are described by Kabalka et al (2002 Nucl Med Biol; 29: 841-843; and 2003 Nuc Med Biol; 30: 369-373). Suitable organotrifluoroborates and their preparation are described by Kabalka et al (2004 Nucl Med Biol; 31: 935-938).

Examples of aryl groups to which radioactive iodine can be attached are given below:

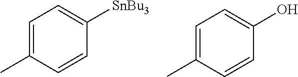

Both contain substituents which permit facile radioiodine substitution onto the aromatic ring. A tyrosine residue permits radioiodination to be carried out using its inherent phenol group.

Alternative substituents containing radioactive iodine can be synthesised by direct radioiodination via radiohalogen exchange, e.g.

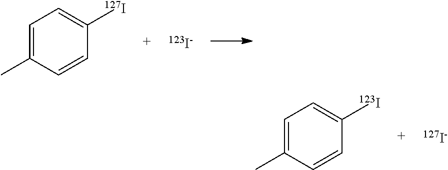

The radioiodine atom is preferably attached via a direct covalent bond to an aromatic ring such as a benzene ring, or a vinyl group since it is known that iodine atoms bound to saturated aliphatic systems are prone to in vivo metabolism and hence loss of the radioiodine.

Making reference to Scheme 1 above, a precursor compound suitable for radioiodination can be obtained starting with compound 7 where one of R²²-R²⁴ of is a bromo group. This compound can be reacted with a suitable stannane to obtain a trialkyltin precursor compound that may be reacted with radioiodine to obtain a radioiodinated in vivo imaging agent of Formula I. Alternatively, one of R²²-R²⁴ of compound 7 can be ¹²⁷I, and this is a precursor compound suitable for radioiodination by radiohalogen exchange.

Precursor compounds suitable for radiofluorination may be designed to be directly labelled with [¹⁸F]-Fluoride, or to be reactive with a ¹⁸F-containing prosthetic group.

Radiofluorination may be carried out via direct labelling using the reaction of [¹⁸F]-Fluoride with a suitable precursor group in the precursor compound of Formula II. The precursor group may be a good leaving group, such as an alkyl bromide, alkyl mesylate or alkyl tosylate. Direct radiofluorination with [¹⁸F]-fluoride may also be carried out by nucleophilic aromatic substitution. For aryl systems, ¹⁸F-fluoride nucleophilic displacement from an aryl diazonium salt, aryl nitro compound or an aryl quaternary ammonium salt are suitable routes to aryl-¹⁸F derivatives. Alternatively, the precursor compound may contain a chloro nicotinamide precursor group where ¹⁸F-fluoride nucleophilic displacement at the chloro leads to the [¹⁸F]fluoronicotinamide compounds (Greguric et al 2009 J Med Chem; 52: 5299-5302). Preferably where direct labelling is carried out with [¹⁸F]-Fluoride, the precursor group is present at R¹⁵ of Formula II. Such precursor compounds may be obtained by starting with compound 7 of Scheme 1 wherein Y²¹ is —X²¹—R²⁵ where X²¹ is N and R²⁵ is a protecting group. After removal of this R²⁵ protecting group, coupling reactions can be carried out at the free amino group.

For example, a reactive derivative of the precursor group R¹⁵ may be used to introduce an R¹⁵ precursor group. Alternatively, an optionally-protected reactive derivative of the R¹⁶ linker may be coupled followed by deprotection and reaction with a reactive derivative of the R¹⁷ precursor group.

¹⁸F can also be introduced by reaction of the precursor compound with an ¹⁸F-labelled prosthetic group. For example, an N-haloacetyl precursor group can be alkylated with the prosthetic group ¹⁸F(CH₂)₃OH to give —NH(CO)CH₂O(CH₂)₃¹⁸F derivatives. A ¹⁸F-labelled compound of the invention may also be obtained by formation of ¹⁸F fluorodialkylamines and subsequent amide formation when the ¹⁸F fluorodialkylamine is reacted with a precursor compound containing a precursor group selected from e.g. chlorine, P(O)Ph₃ or an activated ester. A [¹⁸F]-N-methylaminooxy-containing prosthetic group (Olberg et al Bioconjugate Chem 2008; 19: 1301-1308) may be reacted with a vinylsulphonyl precursor group in the precursor compound to obtain the ¹⁸F-labelled in vivo imaging agent. Another suitable prosthetic group is 4-[¹⁸F]fluorobenzaldehyde, which is obtained as described in WO2004080492, and reacts with NH₂ to form —N═CH—[¹⁸F]fluorophenyl derivatives. The [¹⁸F]fluoronicotinamide compounds described in the previous paragraph can also be obtained by reaction of a precursor compound having a free amino group with [¹⁸F]-6-fluoronicotinic acid tetrafluorophenyl ester, as described in co-pending patent application number GB0905438.8. As described therein, this reaction may be effected in a suitable solvent, for example, in an aqueous buffer in the pH range 2 to 11, suitably 3 to 11, and at a non-extreme temperature of from 5 to 70° C., preferably at ambient temperature.

Further details of synthetic routes to ¹⁸F-labelled derivatives are described by Bolton (2002 J Lab Comp Radiopharm; 45: 485-528).

To obtain in vivo imaging agents of the invention comprising a reporter suitable for optical imaging, the reader is directed to the methods described by Licha et al (2002 Topics Curr Chem; 222: 1-29; 2005 Adv Drug Deliv Rev; 57: 1087-1108). For reviews and examples of labelling using fluorescent dye labelling reagents, see “Non-Radioactive Labelling, a Practical Introduction”, Garman, A. J. Academic Press, 1997; “Bioconjugation—Protein Coupling Techniques for the Biomedical Sciences”, Aslam, M. and Dent, A., Macmillan Reference Ltd, (1998). Cyanine dyes (Cy^{D}) functionalised suitable for conjugation are commercially available from GE Healthcare Limited, Atto-Tec, Dyomics, Molecular Probes and others. Most such dyes are available as NHS esters, which can react with an amine in the precursor compound of Formula II as defined herein to form the desired in vivo imaging agent. Alexa Fluor™ 647 functionalised with hydrazide, maleimide or succinimidyl ester groups are commercially-available from Molecular Probes. Cy^{D} functionalised with carboxyl or maleimide groups can be prepared according to methods described in EP1816475. These functionalised Cy^{D} compounds can be reacted with precursor compounds comprising hydroxy or amine precursor groups to result in the desired in vivo imaging agent of Formula I. A preferred location for a Cy^{D} is at R⁵ of Formula I.

The method for preparation of the in vivo imaging agent of Formula I may also be carried out by solid phase synthesis. A “solid phase” is a cross linked, insoluble polymeric material that is chemically inert to the conditions of the synthesis. The solid phase typically takes the form of spherical particles e.g. beads of diameter between 0.04-0.15 mm, but sheets, pin-shaped particles and disc-shaped particles are also used.

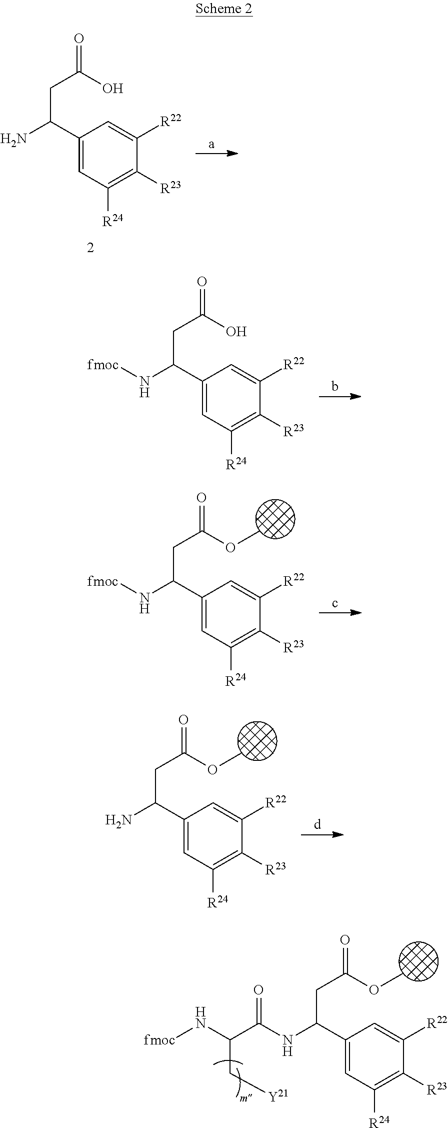

R²²-R²⁴, Y²¹ and m″ are as suitably and preferably defined above for Scheme 1. The solid phase is represented in Scheme 2 by a cross-hatched circle. The starting compound is compound 2 from Scheme 1, which is Fmoc protected in step (a) and then attached to the solid phase in step (b) followed by removal of the Fmoc protecting group in step (c), e.g. by 20% piperidine in dimethyl formamide or NMP. In step (d) the solid-phase compound 2 is reacted with Fmoc-NH—CH((CH₂)_{m′″}—Y²¹. Fmoc is cleaved as described above, and steps analogous to (d)-(f) of Scheme 1 are then carried out on what is a solid-phase version of compound 5 of Scheme 1. The whole precursor compound can be synthesized on the solid phase, and cleaved off by standard methods, for example trifluoroacetic acid in dichloromethane. Alternatively, depending on the chemistries involved, an intermediate can be cleaved off at a suitable stage and subsequent steps run in solution.

In a preferred embodiment, the method for preparation of the in vivo imaging agent of the invention is automated. Automated synthesis may be conveniently carried out by means of an automated synthesis apparatus, e.g. Tracerlab™ and Fastlab™ (both available from GE Healthcare). Fastlab™ represents the state of the art in automated positron-emission tomography (PET) radiotracer synthesis platforms, and it is desirable in the development of a new PET radiotracer that its synthesis is compatible with Fastlab™. The radiochemistry is performed on the automated synthesis apparatus by fitting a “cassette” to the apparatus. Such a cassette normally includes fluid pathways, a reaction vessel, and ports for receiving reagent vials as well as any solid-phase extraction cartridges used in post-radiosynthetic clean up steps.

In a further aspect of the present invention there is therefore provided a cassette for carrying out the automated method of the invention comprising:
(i) a vessel containing a precursor compound, wherein said precursor compound is as suitably and preferably defined above for the method of the invention; and,
(ii) means for eluting the vessel with a suitable source of an in vivo imaging moiety, said in vivo imaging moiety as suitably and preferably defined herein.

The cassette may also comprise an ion-exchange cartridge for removal of excess in vivo imaging moiety. The reagents, solvents and other consumables required for the automated synthesis may also be included together with a data medium, such as a compact disc carrying software, which allows the automated synthesiser to be operated in a way to meet the end user's requirements for concentration, volumes, time of delivery etc. The cassette of the invention is particularly suitable for preparation of in vivo imaging agents of the invention where the in vivo imaging moiety is ¹⁸F.

In a further aspect, the present invention provides a “pharmaceutical composition”, which is defined as a composition comprising the in vivo imaging agent as defined herein together with a biocompatible carrier, in a form suitable for mammalian administration.

The “biocompatible carrier” is a fluid, especially a liquid, in which the in vivo imaging agent is suspended or dissolved, such that the composition is “suitable for mammalian administration”, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

The pharmaceutical composition of the invention is suitably supplied in a container which is provided with a seal which is suitable for single or multiple puncturing with a hypodermic needle (e.g. a crimped-on septum seal closure) whilst maintaining sterile integrity. Such containers may contain single or multiple patient doses. Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. Where the pharmaceutical composition is a radiopharmaceutical composition, the pre-filled syringe may optionally be provided with a syringe shield to protect the operator from radioactive dose. Suitable such radiopharmaceutical syringe shields are known in the art and preferably comprise either lead or tungsten.

The pharmaceutical composition of the present invention may be prepared from a kit. Alternatively, the pharmaceutical composition may be prepared under aseptic manufacture conditions to give the desired sterile product. The pharmaceutical composition may also be prepared under non-sterile conditions, followed by terminal sterilisation using e.g. gamma-irradiation, autoclaving, dry heat or chemical treatment (e.g. with ethylene oxide). Preferably, the pharmaceutical composition of the present invention is prepared from a kit.

Such kits comprise a suitable precursor of the invention, preferably in sterile non-pyrogenic form, so that reaction with a sterile source of an in vivo imaging moiety gives the desired pharmaceutical composition with the minimum number of manipulations. Where the kit comprises a precursor compound of the invention, said kit itself forms a further aspect of the invention. Such considerations are particularly important for radiopharmaceuticals, especially where the radioisotope has a relatively short half-life, and for ease of handling and hence reduced radiation dose for the radiopharmacist. Hence, the reaction medium for reconstitution of such kits is preferably a biocompatible carrier as defined above, and is most preferably aqueous. The precursors for use in the kit may be employed under aseptic manufacture conditions to give the desired sterile, non-pyrogenic material. The precursors may also be employed under non-sterile conditions, followed by terminal sterilisation using e.g. gamma-irradiation, autoclaving, dry heat or chemical treatment (e.g. with ethylene oxide). Preferably, the precursors are employed in sterile, non-pyrogenic form.

The kits may optionally further comprise additional components such as a radioprotectant, antimicrobial preservative, pH-adjusting agent or filler.

By the term “radioprotectant” is meant a compound which inhibits degradation reactions, such as redox processes, by trapping highly-reactive free radicals, such as oxygen-containing free radicals arising from the radiolysis of water. Suitable radioprotectants are chosen from: ascorbic acid, para-aminobenzoic acid (i.e. 4-aminobenzoic acid), gentisic acid (i.e. 2,5-dihydroxybenzoic acid) and salts thereof with a biocompatible cation.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the pharmaceutical composition post-reconstitution, i.e. in the imaging agent product itself. The antimicrobial preservative may, however, also optionally be used to inhibit the growth of potentially harmful micro-organisms in one or more components of the kit prior to reconstitution. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS (i.e. tris(hydroxymethyl)aminomethane), and pharmaceutically acceptable bases such as sodium carbonate, sodium bicarbonate or mixtures thereof.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

In a yet further aspect, the present invention provides an in vivo imaging method to determine the quantity and/or location of αᵥβ₆ expressed in a subject, wherein said method comprises:
(i) administering to said subject the in vivo imaging agent as suitably and preferably defined herein;
(ii) allowing said administered in vivo imaging agent of step (i) to bind to αᵥβ₆ expressed in said subject;
(iii) detecting signals emitted by an in vivo imaging moiety comprised in said bound in vivo imaging agent of step (ii); and,
(iv) generating an image of the location and amount of said signals, wherein said signals represent the quantity and/or location of αᵥβ₆ expressed in said subject.

The “subject” of the invention can be any human or animal subject. Preferably the subject of the invention is a mammal. Most preferably, said subject is an intact mammalian body in vivo. In an especially preferred embodiment, the subject of the invention is a human.

The step of “administering” the in vivo imaging agent is preferably carried out parenterally, and most preferably intravenously. The intravenous route represents the most efficient way to deliver the in vivo imaging agent throughout the body of the subject and into contact with αᵥβ₆ expressed in said subject. The in vivo imaging agent of the invention is preferably administered as the pharmaceutical composition of the invention, as defined herein.

Following the administering step and preceding the detecting step, the in vivo imaging agent is allowed to bind to αᵥβ₆. The in vivo imaging agent moves dynamically through the subject's body, coming into contact with various tissues therein. Once the in vivo imaging agent comes into contact with αᵥβ₆, a specific interaction takes place such that clearance of the in vivo imaging agent from tissue with αᵥβ₆ takes longer than from tissue without, or expressing less αᵥβ₆. A certain point in time is reached when detection of in vivo imaging agent specifically bound to αᵥβ₆ is enabled as a result of the ratio between in vivo imaging agent bound to tissue with αᵥβ₆ versus that bound in tissue expressing less (or no) αᵥβ₆.

The step of “detecting signals” involves detection of signals emitted by the in vivo imaging moiety by means of a detector sensitive to said signals. Such detectors are well-known in the art. For example, where the in vivo imaging moiety is a gamma emitter, detection can be carried out using a single-photon emission computed tomography (SPECT) camera, and where the in vivo imaging moiety is a paramagnetic metal ion, detection can be carried out using a magnetic resonance imaging (MRI) camera.

The step of “generating an image” is carried out by a computer which applies a reconstruction algorithm to the acquired signal data to yield a dataset. This dataset is then manipulated to generate an image showing the location and/or amount of signals emitted by said in vivo imaging moiety.

An “αᵥβ₆ condition” means a pathological condition characterised by abnormal expression of αᵥβ₆, typically over-expression of αᵥβ₆. Examples of such conditions where the method of in vivo imaging of the present invention would be of use include inflammation, cancer and fibrosis. The in vivo imaging method of the invention is preferably carried out wherein said subject is known or is suspected to have an αᵥβ₆ condition, most preferably said subject is known to have an αᵥβ₆ condition. Where the subject is known to have an αᵥβ₆ condition, said in vivo imaging method is preferably carried out repeatedly during the course of a treatment regimen for said subject, said treatment regimen comprising administration of a drug to combat said αᵥβ₆ condition.

In a preferred embodiment, the in vivo imaging method as suitably and preferably defined herein further comprises the step (v) of attributing the quantity and/or location of αᵥβ₆ receptors to a particular clinical picture.

In a yet further aspect, the present invention provides the in vivo imaging agent as suitably and preferably defined herein for use in the in vivo imaging method as suitably and preferably defined herein.

The invention will now be described in the following non-limiting examples.

### BRIEF DESCRIPTION OF THE EXAMPLES

Example 1 describes the synthesis of non-radioactive imaging agent 1.

Example 2 describes the synthesis of imaging agent 2.

Example 3 describes the synthesis of imaging agent 3.

Example 4 describes the synthesis of non-radioactive imaging agent 4.

Example 5 describes the synthesis of imaging agent 4.

Example 6 describes the synthesis of imaging agent 5.

Example 7 describes the synthesis of imaging agent 6.

Example 8 describes an in vitro affinity assay for assessment of αᵥβ₃ binding.

Example 9 describes the synthesis of imaging agent 7.

Example 10 describes a flow cytometry evaluation of imaging agent 7.

### LIST OF ABBREVIATIONS USED IN THE EXAMPLES

aq aqueous

Boc t-Butyloxycarbonyl

Bu butyl

Bzl benzyl

Dpr diaminopropionic acid

ESI-MS electrospray ionisation mass spectrometry

Fmoc 9-Fluorenylmethyloxycarbonyl

h hour(s)

LC-MS liquid chromatography-mass spectrometry

MDP methylenediphosphonic acid

Me methyl

MH+ molecular ion

min minute(s)

m/z mass-to-charge ratio

PEG polyethyleneglycol

Ser serine

TFA trifluoroacetic acid

t_{R} retention time

trityl trimethyl phenyl

UV ultraviolet

### Examples

### Example 1

### Synthesis of 3-(3-(benzyloxy)-2-(5-(pyridin-2-ylamino)pentanamido)-propanamido)-3-(3-chloro-5-iodophenyl)propanoic acid (Non-radioactive Imaging agent 1)

### Example 1(i)

### Synthesis of 3-(Fmoc-amino)-3-(3-bromo-5-chlorophenyl)propanoic acid

The compound was synthesised as described in Example 2(i) below but starting with 3-bromo-5-chlorobenzaldehyde (ABCR, 3.0 g, 14 mmol). Yield 1.3 g (19% over two steps). LC-MS (column Phenomenex Luna C18(2) 20×2 mm, 3 μm, solvents: A=water/0.1% trifluoroacetic acid and B=acetonitrile/0.1% trifluoroacetic acid; gradient 05-80% B over 5 min; flow rate 0.6 mL/min, UV detection at 214 and 254 nm, ESI-MS) t_{R}=4.1 min, m/z expected 500.0 (Ma), found 500.0.

### Example 1(ii)

### Synthesis of 3-(3-(benzyloxy)-2-(5-(pyridin-2-ylamino)pentanamido)propanamido)-3-(3-bromo-5-chlorophenyl)propanoic acid

The compound was synthesised on solid support using the same method as described in Examples 2(ii), 2(iii), 2(vi) and 2(vii) below but starting with attachment of 3-(Fmoc-amino)-3-(3-bromo-5-chlorophenyl)propanoic acid (described in Example 1(i), 0.6 g, 1.2 mmol) on trityl chloride resin (Novabiochem, substitution 1.6 mmol/g, 0.5 g) followed by coupling of Fmoc-Ser(Bzl)-OH (Aldrich, 0.5 g, 1.2 mmol) and 5-((tert-butoxycarbonyl(pyridin-2-yl)amino)pentanoic acid (described in Example 2(v) below, 0.21 g, 0.71 mmol), respectively. Boc de-protection and cleavage from solid support gave 0.211 g crude material.

### Example 1(iii)

### Synthesis of methyl 3-(3-(benzyloxy)-2-(5-(pyridin-2-ylamino)pentanamido)propanamido)-3-(3-bromo-5-chlorophenyl)propanoate

Thionylchloride (Sigma Aldrich, 79 mg, 0.66 mmol) was added slowly to solution of 3-(3-(benzyloxy)-2-(5-(pyridin-2-ylamino)pentanamido)propanamido)-3-(3-bromo-5-chlorophenyl)propanoic acid (described in Example 1(ii), 0.167 g, 0.264 mmol) in methanol (3 mL) at 0° C. (ice/water bath). The mixture was warmed up to room temperature and after 45 minutes quenched with water. The solution was evaporated and the product was re-crystallized from methanol affording 0.155 g (91%).

### Example 1(iv)

### Synthesis of methyl 3-(3-(benzyloxy)-2-(5-(pyridin-2-ylamino)pentanamido)propanamido)-3-(3-chloro-5-(trimethylstannyl)phenyl)propanoate

The reaction was run in a microwave instrument (Biotage). A mixture of methyl 3-(3-(benzyloxy)-2-(5-(pyridin-2-ylamino)pentanamido)propanamido)-3-(3-bromo-5-chlorophenyl)propanoate (described in Example 1(iii), 10 mg, 15 μmol), tetrakis(triphenylphosphine)palladium(0) (Strem chemicals, 1 mg, 0.8 μmol) and hexamethylditin (Sigma-Aldrich, 40.0 mg, 122 μmol) in toluene (2 mL) was purged with nitrogen and heated at 140° C. for 30 min. Purification by flash chromatography, (silica, dichloromethane/methanol, 9:1, with 1% triethylamine) afforded 5 mg semipure product.

### Example 1(v)

### Synthesis of methyl 3-(3-(benzyloxy)-2-(5-(pyridin-2-ylamino)pentanamido)propanamido)-3-(3-chloro-5-iodophenyl)propanoate

A solution of iodine (Fluka, 2 mg, 7 μmol) and methyl 3-(3-(benzyloxy)-2-(5-(pyridin-2-ylamino)pentanamido)propanamido)-3-(3-chloro-5-(trimethylstannyl)phenyl)propanoate (described in Example 1(iv), 5 mg, 7 μmol) in dichloromethane (2 mL) was stirred at room temperature for 2 h. The reaction mixture was treated with sodium thiosulphate solution (0.3 M, 1 mL). After separation of the phases the dichloromethane solution was washed with water and brine, dried (sodium sulphate) and concentrated to give 4 mg of crude material.). LC-MS (column Phenomenex Luna C18(2) 20×2 mm, 3 μm, solvents: A=water/0.1% trifluoroacetic acid and B=acetonitrile/0.1% trifluoroacetic acid; gradient 10-80% B over 5 min; flow rate 0.6 mL/min, UV detection at 214 and 254 nm, ESI-MS) t_{R}=2.8 min, m/z expected 693.1 (MH⁺), found 693.3.

### Example 1(vi)

### Synthesis of 3-(3-(benzyloxy)-2-(5-(pyridin-2-ylamino)pentanamido)propanamido)-3-(3-chloro-5-iodophenyl)propanoic acid

Methyl 3-(3-(benzyloxy)-2-(5-(pyridin-2-ylamino)pentanamido)propanamido)-3-(3-chloro-5-iodophenyl)propanoate (described in Example 1(v), 4 mg, 6 μmol) was dissolved in a mixture of methanol (1 mL) and aq sodium hydroxide (1 M, 0.2 mL). The reaction mixture was stirred at room temperature for 1 h. The solution was extracted with dichloromethane (2×2 mL). The aqueous phase was adjusted to pH 6 with hydrochloric acid (1 M) and then extracted with dichloromethane (2×2 mL) and ethyl acetate (2×2 mL). The organic phases were combined and dried (sodium sulphate), filtered and concentrated to give 0.5 mg. LC-MS (column Phenomenex Luna C18(2) 20×2 mm, 3 μm, solvents: A=water/0.1% trifluoroacetic acid and B=acetonitrile/0.1% trifluoroacetic acid; gradient 5-80% B over 5 min; flow rate 0.6 mL/min, UV detection at 214 and 254 nm, ESI-MS) t_{R}=2.8 min, m/z expected 679.1 (MH⁺), found 679.2.

### Example 2

### Synthesis of (6S,Z)-3-(3,5-dichlorophenyl)-22-(hydroxyimino)-17-(2-(((Z)-3-(hydroxyimino)-2-methylbutan-2-yl)amino)ethyl)-21,21-dimethyl-5,9,13-trioxo-6-(5-(pyridin-2-ylamino)pentanamido)-4,8,14,20-tetraazatricosan-1-oic acid (Imaging Agent 2)

### Example 2(i)

### Synthesis of Fmoc-3-amino-3-(3,5-dichlorophenyl)propanoic acid

To a stirred suspension of 3-amino-3-(3,5-dichlorophenyl)propanoic acid (synthesised as described in J Med Chem; 45:1045-1051 and Tetrahedron Asym; 19: 2072-2077) (100 mg, 0.43 mmol) in chloroform/methanol/water (65:25:4; 5 mL) was added diisopropylethylamine (Fluka, 227 μL, 1.30 mmol). To the solution was added 9-fluorenylmethoxycarbonyl-N-hydroxysuccinimide (Novabiochem, 169 mg, 0.50 mmol). After 1 h the mixture was concentrated (rotavapor) and the residue was take up in acetonitrile (10 mL) containing 0.1% trifluoroacetic acid, stirred for 5 min and filtered. The solution was concentrated and the residue was subjected to flash chromatography. The solid material was applied directly on the column and eluted with hexane/ethyl acetate (1:1) to remove impurities followed by elution of the product with chloroform/methanol (9:1), affording 177 mg (91%) of the product as colourless oil. LC-MS (column Phenomenex Luna C18(2) 20×2 mm, 3 μm, solvents: A=water/0.1% trifluoroacetic acid and B=acetonitrile/0.1% trifluoroacetic acid; gradient 5-80% B over 5 min; flow rate 0.6 mL/min, UV detection at 214 and 254 nm, ESI-MS) t_{R}=4.1 min, m/z expected 456.1 (MH⁺), found 456.1.

### Example 2(ii)

### Attachment of Fmoc-3-amino-3-(3,5-dichlorophenyl)propanoic acid to solid support

To trityl chloride resin (Novabiochem, substitution 1.6 mmol/g, 0.406 g) was added a solution of Fmoc-3-amino-3-(3,5-dichlorophenyl)propanoic acid (described in Example 2(i), 0.30 g, 0.65 mmol) in a mixture of dichloromethane and N-methylpyrrolidone (8:1; 9 mL) followed by addition of diisopropylethylamine (Fluka, 2 M solution in N-methylpyrrolidone, 2.6 mL, 5.2 mmol). The mixture was kept at a roller table for 5 h and then transferred to a manual nitrogen bubbler apparatus. The resin was drained and treated with a dichloromethane/methanol/diisopropylethylamine solution (17:2:1; 6 mL) three times, washed with dichloromethane and dried.

### Example 2(iii)

### Coupling of Fmoc-Dpr-(ivDde)-OH

The reaction was carried out in a manual nitrogen bubbler apparatus on the resin described in Example 2(ii) (0.65 mmol). The Fmoc group was removed by standard treatment (20% piperidine in N-methylpyrrolidone). Fmoc-Dpr(ivDde)-OH (Novabiochem, 0.69 g, 1.3 mmol) was preactivated for 5 min with HATU ((N-[(dimethylamino)-1H-1,2,3-triazolo-[4,5-b]pyridin-1-ylmethylene]-N-methylmethanaminium hexafluorophosphate N-oxide, Genscript Corp, 0.49 g, 1.3 mmol) and diisopropylethylamine (Fluka, 2 M solution in N-methylpyrrolidone, 1.3 mL, 2.6 mmol) in N-methylpyrrolidone (7 mL) and added to the resin. After 2 h the resin was drained and washed with N-methylpyrrolidone. Standard Kaiser test indicated incomplete reaction and the coupling was repeated. An aliquot of the resin was cleaved (5% trifluoroacetic acid in dichloromethane, 5 min) and analysed by LC-MS (column Phenomenex Luna C18(2) 20×2 mm, 3 μm, solvents: A=water/0.1% trifluoroacetic acid and B=acetonitrile/0.1% trifluoroacetic acid; gradient 10-80% B over 5 min; flow rate 0.6 mL/min, UV detection at 214 and 254 nm, ESI-MS) t_{R}=4.4 min, m/z expected 748.3 (MH⁺), found 748.2.

### Example 2(iv)

### 5-(tert-Butoxycarbonyl-pyridin-2-yl-amino)pentanoic acid methyl ester

A solution of 2-(N-(tert-butoxycarbonyl)amino)pyridine (1.0 g, 5.2 mmol, synthesised as described in J Org Chem; 67: 4965-4967) in anhydrous dimethylformamide (15 mL) was cooled to 5° C. in ice/water bath. Sodium hydride (Aldrich, 60% in mineral oil, 0.25 g, 6.3 mmol) was added in portions. The cooling bath was removed and the mixture stirred vigorously for 20 min. The mixture was then cooled (ice/water bath) and methyl 5-bromovalerate (Aldrich, 0.89 mL, 6.3 mmol) was added drop wise. After 10 min the cooling bath was removed and the mixture was stirred for 2 h and then concentrated (rotavapor). The residue was taken up in a mixture of dichloromethane (30 mL) and hydrochloric acid (0.02 N, 20 mL) and shaken vigorously. The phases were separated and the organic phase was extracted with saturated aqueous sodium bicarbonate (20 mL) and brine (20 mL) and dried (Na₂SO₄). After concentration (rotavapor) the crude material was obtained as colourless oil (1.52 g). An aliquot of crude material (0.5 g) was purified by flash chromatography (hexane/ethyl acetate, 1:1) affording the product as colourless oil, 490 mg yield (93%). LC-MS (column Phenomenex Luna C18(2) 20×2 mm, 3 μm, solvents: A=water/0.1% trifluoroacetic acid and B=acetonitrile/0.1% trifluoroacetic acid; gradient 5-80% B over 5 min; flow rate 0.6 mL/min, UV detection at 214 and 254 nm, ESI-MS) t_{R}=2.6 min, m/z expected 309.2 (MH⁺), found 309.4.

### Example 2(v)

### 5-(tert-Butoxycarbonyl-pyridin-2-yl-amino)pentanoic acid

To a stirred solution of the ester described in Example 2(iv) (0.69 g, 2.3 mmol) in methanol (10 mL) was added sodium hydroxide (4 N, 2 mL) at ambient temperature. The reaction was monitored by thin layer chromatography (silica, hexane/ethyl acetate, 1:1). After 3 h the solvent was evaporated (rotavapor). Water (8 mL) was added to the residue and the aqueous solution was extracted with ether (2×10 mL), then made slightly acidic (pH ˜5) using hydrochloric acid (2 N) before extraction with dichloromethane (3×10 mL). The combined dichloromethane phases were dried (Na₂SO₄), filtered and evaporated to afford the product as colourless oil, 594 mg yield (90%). LC-MS (column Phenomenex Luna C18(2) 20×2 mm, 3 μm, solvents: A=water/0.1% trifluoroacetic acid and B=acetonitrile/0.1% trifluoroacetic acid; gradient 5-80% B over 5 min; flow rate 0.6 mL/min, UV detection at 214 and 254 nm, ESI-MS) t_{R}=2.0 min, m/z expected 295.2 (MH⁺) found 295.4.

### Example 2(vi)

### Coupling of 5-(tert-butoxycarbonyl(pyridin-2-yl)amino)pentanoic acid

The reaction was carried out in a manual nitrogen bubbler apparatus. The Fmoc group of the resin described in Example 2(iii) was removed by standard treatment (20% piperidine in N-methylpyrrolidone). 5-(tert-Butoxycarbonyl(pyridin-2-yl)amino)pentanoic acid (described in Example 2(v), 0.25 g, 0.85 mmol) was preactivated for 5 min with HATU (Genscript Corp, 0.32 g, 0.85 mmol) and diisopropylethylamine (Fluka, 2 M solution in N-methylpyrrolidone, 0.845 mL, 1.69 mmol) in N-methylpyrrolidone (6 mL) and added to the resin. After 3 h the resin was drained and washed. As Kaiser test indicated incomplete conversion the coupling was repeated. An aliquot of the resin was cleaved (5% trifluoroacetic acid in dichloromethane, 5 min) and analysed by LC-MS (column Phenomenex Luna C18(2) 20×2 mm, 3 μm, solvents: A=water/0.1% trifluoroacetic acid and B=acetonitrile/0.1% trifluoroacetic acid; gradient 10-80% B over 5 min; flow rate 0.6 mL/min, UV detection at 214 and 254 nm, ESI-MS) t_{R}=3.5 min, m/z expected 802.3 (MH⁺), found 802.3.

### Example 2(vii)

### Removal of protection groups and cleavage from solid support

The reaction was carried out in a manual nitrogen bubbler apparatus. To the resin described in Example 2(vi) (0.325 mmol) was added a solution of 2% hydrazine in N-methylpyrrolidone (5 mL). After 2 min the resin was drained and the treatment was repeated twice, with 5 min and 10 min reaction time, respectively. The resin was washed with N-methylpyrrolidone and dichloromethane. Simultaneous removal of the Boc group and cleavage of the product from the resin was done in a trifluoroacetic acid/dichloromethane/triisopropyl silane solution (49:49:2; 6.2 mL). After 2.5 h the resin was filtered off and the solution was concentrated (rotavapor) to dryness. Analysis by LC-MS (column Phenomenex Luna C18(2) 20×2 mm, 3 μm, solvents: A=water/0.1% trifluoroacetic acid and B=acetonitrile/0.1% trifluoroacetic acid; gradient 10-80% B over 5 min; flow rate 0.6 mL/min, UV detection at 214 and 254 nm, ESI-MS) t_{R}=1.5 min, m/z expected 496.1 (MH⁺), found 496.1.

### Example 2(viii)

### Conjugation with cPn216-glutaric acid

cPn216-Glutaric acid (synthesised as described in WO2003006070, 0.26 g, 0.57 mmol) was pre-activated with PyAOP (7-azabenzotriazol-1-yloxy-tris-(pyrrolidino)phosphonium hexafluorophosphate, Applied Biosystems, 0.222 g, 0.426 mmol) and diisopropylethylamine (Aldrich, 148 μL, 0.852 mmol) in dimethylformamide (3 mL) for 10 min. The compound from Example (2(vii) 0.141 g, 0.284 mmol) was dissolved in dimethylformamide (6 mL) and added to the pre-activated chelate. Progress of the reaction was monitored by LC-MS analysis. After 1 h diisopropylethylamine (Fluka, 2M in N-methylpyrrolidone, 0.4 mL, 0.8 mmol) was added and the mixture was stirred for 1 h. A second portion of pre-activated cPn216-glutaric acid (0.143 g, 0.312 mmol) was added (same coupling reagents. After 1 h the reaction was complete and the mixture was concentrated (rotavapor). The compound was purified by preparative HPLC (column Phenomenex Luna C18(2) 10 μm 250×50.0 mm, solvents: A=water/0.1% trifluoroacetic acid and B=acetonitrile/0.1% trifluoroacetic acid; gradient 10-40% B over 60 min; flow 50 mL/min, UV detection at 214 nm and 254 nm) and lyophilised, affording 56 mg (21%). LC-MS (column Phenomenex Luna C18(2) 20×2 mm, 3 μm, solvents: A=water/0.1% trifluoroacetic acid and B=acetonitrile/0.1% trifluoroacetic acid; gradient 10-40% B over 5 min; flow rate 0.6 mL/min, UV detection at 214 and 254 nm, ESI-MS) t_{R}=2.8 min, m/z expected 935.5 (MH⁺), found 935.5.

### Example 2(ix)

### ^{99m}Tc Labelling to Obtain Imaging Agent 2

The precursor compound obtained in Example 2(viii) can be labelled with ^{99m}Tc by incubating at room temperature 100 μg of precursor compound (obtained as described in Example 2(viii)) in methanol with 0.5 ml Na₂CO₃/NaHCO₃ buffer (pH 9.2), 0.5 ml TcO₄⁻ from a ^{99m}Tc generator and 0.1 ml SnCl₂/MDP solution.

### Example 3

### Synthesis of 3-((S)-3-(4-(2-Aminoethylamino)-3-((2-aminoethylamino)methyl)butanamido)-2-(5-(pyridin-2-ylamino)pentanamido)propanamido)-3-(3,5-dichlorophenyl)propanoic acid (Imaging agent 3)

### Example 3(i)

### Coupling of tetra-Boc-tetraamine-NHS ester

The reaction was carried out in a manual nitrogen bubbler apparatus. To the resin described in Example 2(vi) (0.074 mmol) was added a solution of 2% hydrazine in N-methylpyrrolidone (5 mL). After 2 min the resin was drained and the treatment was repeated twice, with 5 min and 10 min reaction time, respectively. The resin was washed with N-methylpyrrolidone and dichloromethane and drained. The resin was suspended in N-methylpyrrolidone followed by addition of the tetra-Boc-tetraamine-NHS ester (synthesised as described in WO2006008496, 0.053 g, 0.074 mmol) and N-methylmorpholine (Merck, 9.7 μL, 0.088 mmol). After 2 h the resin was drained and washed. The coupling was followed by standard Kaiser test and was repeated 3 more times. An aliquot of the resin was cleaved (50% trifluoroacetic acid in dichloromethane, 5 min) and analysed by LC-MS (column Phenomenex Luna C18(2) 20×2 mm, 3 μm, solvents: A=water/0.1% trifluoroacetic acid and B=acetonitrile/0.1% trifluoroacetic acid; gradient 0-60% B over 5 min; flow rate 0.6 mL/min, UV detection at 214 and 254 nm, ESI-MS) t_{R}=3.2 min, m/z expected 348.7 ((M-Boc₅)H₂)²⁺, found 348.6.

### Example 3(ii)

### Boc deprotection and cleavage from solid support

Simultaneous removal of the Boc protecting groups and cleavage of the product from the solid support was achieved by treating the resin described in Example 3(i) (0.074 mmol) with a trifluoroacetic acid/triisopropyl silane/water (1.9 mL, 5 μL, 5 μL) solution for 2 h. The solution was filtered from the resin and concentrated (rotavapor). The product was purified by preparative HPLC (column Phenomenex Luna C18(2) 5 μm 250×21.2 mm, solvents: A=water/0.1% trifluoroacetic acid and B=acetonitrile/0.1% trifluoroacetic acid; gradient 5-50% B over 60 min; flow 10.0 mL/min, UV detection at 214 nm and 254 nm) and lyophilised. Yield 8.1 mg (16%). LC-MS (column Phenomenex Luna C18(2) 20×2 mm, 3 μm, solvents: A=water/0.1% trifluoroacetic acid and B=acetonitrile/0.1% trifluoroacetic acid; gradient 10-80% B over 5 min; flow rate 0.6 mL/min, UV detection at 214 and 254 nm, ESI-MS) t_{R}=2.3 min, m/z expected 696.3 (MH⁺), found 696.3.

### Example 3(iii)

### ^{99m}Tc Labelling to Obtain Imaging Agent 3

The precursor compound obtained in Example 3(ii) can be labelled with ^{99m}Tc using the method described in Example 2(ix).

### Example 4

### (48S,E)-51-(3,5-Dichlorophenyl)-1-(4-fluorophenyl)-5,45,49-trioxo-48-(5-(pyridin-2-ylamino)pentanamido)-3,9,12,15,18,21,24,27,30,33,36,39,42-tridecaoxa-2,6,46,50-tetraazatripentacont-1-en-53-oic acid (Non-radioactive Imaging agent 4)

### Example 4(i)

### Conjugation with Fmoc-PEG(12) propionic acid

The reaction was carried out in a manual nitrogen bubbler apparatus. To the resin described in Example 2(vi) (0.17 mmol) was added 2% hydrazine in N-methylpyrrolidone (10 mL). After 5 min the resin was drained and the treatment was repeated with 15 min reaction time. The resin was washed with N-methylpyrrolidone and dichloromethane. 1-(9H-Fluoren-9-yl)-3-oxo-2,7,10,13,16,19,22,25,28,31,34,37,40-tridecaoxa-4-azatritetracontan-43-oic acid (Polypure, 0.244 g, 0.290 mmol) was preactivated with HATU (Applied Biosystems, 0.11 g, 0.29 mmol) and diisopropylethylamine (Fluka, 50 μL, 0.29 mmol) in N-methylpyrrolidone (2 mL) for 5 min and added to the resin. The coupling was followed by standard Kaiser test. After 2 h the resin was drained and the coupling was repeated using a solution of Fmoc-amino PEG(12) propionic acid (0.134 mg, 0.160 mmol), HATU (0.070 g, 0.18 mmol) and diisopropylethylamine (80 μL, 0.46 mmol) preactivated in N-methylpyrrolidone (2 mL) for 5 min. After 3 h the mixture was drained and washed with N-methylpyrrolidone and dichloromethane. Standard Kaiser test showed complete coupling.

### Example 4(ii)

### Conjugation with Fmoc-aminooxyacetic acid

The reaction was carried out in a manual nitrogen bubbler apparatus. The resin described in Example 4(i) (0.17 mmol) was treated with 20% piperidine in N-methylpyrrolidone for standard removal of Fmoc groups. Fmoc-aminoxyacetic acid (Iris Biotech GmbH, 0.050 g, 0.16 mmol) was preactivated for 5 min with HATU (Applied Biosystems, 0.070 g, 0.18 mmol) and diisopropylethylamine (Fluka, 80 μL, 0.46 mmol) in N-methylpyrrolidone (2 mL) and then added to the resin. After 2 h the resin was drained and washed. Kaiser test indicated incomplete conversion and the coupling was repeated twice using the same conditions.

### Example 4(iii)

### Removal of protection groups and cleavage from solid support

The resin from Example 4(ii) (0.17 mmol) was subjected to standard Fmoc deprotection using 20% piperidine in N-methylpyrrolidone in a manual nitrogen bubbler apparatus. The resin was washed with N-methylpyrrolidone, dichloromethane and drained. Simultaneous removal of the Boc protecting group and cleavage of the material from the resin was performed using a mixture of trifluoroacetic acid (3 mL), dichloromethane (3 mL) and triisopropyl silane (150 μL). After 2.5 h the solution was filtered and concentrated (rotavapor). LC-MS (column Phenomenex Luna C18(2) 20×2 mm, 3 μm, solvents: A=water/0.1% trifluoroacetic acid and B=acetonitrile/0.1% trifluoroacetic acid; gradient 5-40% B over 5 min; flow rate 0.6 mL/min, UV detection at 214 and 254 nm, ESI-MS) t_{R}=3.5 min, m/z expected 584.8 (MH₂)²⁺, found 584.3.

### Example 4(iv)

### Oxime formation with 4-fluorobenzaldehyde (Non-radioactive Imaging agent 4)

To a solution of the product described in Example 4(iii) (0.17 mmol) in water/acetonitrile (4:1; 3 mL) was added 4-fluorobenzaldehyde (Fluka, 4 μL, 0.04 mmol). The reaction mixture was stirred at 70° C. for 1 h. The mixture was concentrated and the product purified by preparative HPLC (column Phenomenex Luna C18(2) 5 μm 21.2×250 mm, solvents: A=water/0.1% trifluoroacetic acid and B=acetonitrile/0.1% trifluoroacetic acid; gradient 10-80% B over 60 min; flow 10.0 ml/min, UV detection at 214 nm and 254 nm) affording, 11 mg yield (5%) after lyophilisation. LC-MS (column Phenomenex Luna C18(2) 20×2 mm, 3 μm, solvents: A=water/0.1% trifluoroacetic acid and B=acetonitrile/0.1% trifluoroacetic acid; gradient 10-80% B over 5 min; flow rate 0.6 mL/min, UV detection at 214 and 254 nm, ESI-MS) t_{R}=2.3 min, m/z expected 637.8 (MH₂)²⁺, found 637.4.

### Example 5

### Synthesis of Imaging Agent 4

The commercially-available benzaldehyde (available from Sigma-Aldrich) is reacted step (a) with malonic acid and ammonium acetate, and then with methanol and SOCl₂ in step (b) according to the method of Goodman et al, supra. Using standard peptide coupling reagents and methods, Boc-Dpr(Fmoc)-OH is reacted in step (c) with the product of step (b), followed by deprotection to remove the Boc protecting group in step (d) using 2M HCl in dioxane. The Boc-deprotected compound is then reacted in step (e) with 5-(pyridin-2-yl)aminopentanoic acid (obtained as described by Goodman et al, supra). In step (f) the ester group is saponified and the Fmoc side chain is cleaved using NaOH (aq)/dioxane. The compound obtained in step (f) is reacted in step (g) with Boc-amino-PEG-carboxylic acid (from Polypure) preactivated with standard peptide coupling reagents. In step (h) the compound obtained in step (g) is reacted with (i) HCl in dioxane or TFA in dichloromethane, (ii) preactivated Boc-aminooxyacetic acid, and (iii) HCl in dioxane or TFA in dichloromethane to result in the precursor compound. This precursor compound can be labelled with ¹⁸F by reaction with 4-[¹⁸F]fluorobenzaldehyde (the synthesis of which is described in WO2004080492) to result in in vivo imaging agent 4.

### Example 6

### Synthesis of Imaging Agent 5

The starting compound, obtained as described in Example 5, is reacted in step (a) with Vinylsulphonylacetic acid/coupling reagent/base (Scöberl and Biederman 1968 Liebigs Ann Chem; 716: 37-46; Olberg et al 2009 J Labelled Compd Radiopharm; published online 2009). In steps (b) diethylene glycol is converted to the ¹⁸F labelled prosthetic group in three steps, followed by conjugation in step (c), following the methods described by Olberg et al 2008 (Bioconjugate Chem; 19: 1301-1308) and Olberg et al 2009 (J Labelled Compd Radiopharm; published online)

### Example 7

### Synthesis of Imaging Agent 6

The starting compound, obtained as described in Example 5, is reacted in step (a) with the Boc-protected amino-PEG, followed by deprotection in step (b) using e.g. TFA in dichloromethane or HCl in dioxane to result in the precursor compound. ¹⁸F labeling can be carried out using either the two-step process of steps (c) and (d), i.e. reaction with 6-chloronicotinic acid in step (c) followed by reaction with [¹⁸F]Fluoride in step (d) (following methods described by Hocke et al 2005 Bioorg Med Chem Lett; 15: 4819 and by Greguric et al 2009 J Med Chem; 52: 5299). Alternatively, ¹⁸F labelling may be carried out in one step (e) by reaction of the precursor compound with [¹⁸F]-6-fluoronicotinic acid tetrafluorophenyl ester, as described in co-pending patent application number GB0905438.8. As described therein, this reaction may be effected in an aqueous buffer in the pH range 2 to 11 and at ambient temperature.

### Example 8

### In vitro affinity assessment αvβ3

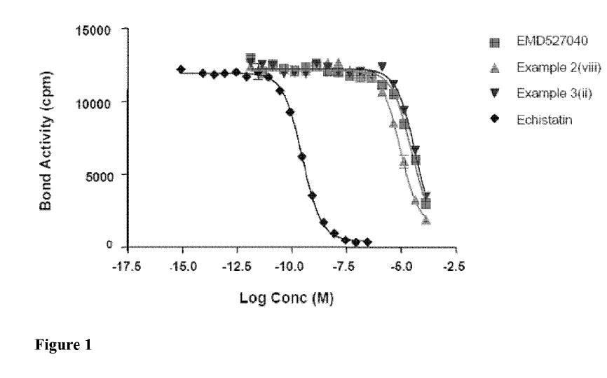

Affinity for integrin receptors αvβ3 was measured by competition experiments with [¹¹⁵I]-Echistatin on membrane fractions prepared from human endothelial adenocarcinoma cell line EA-Hy 926 using a homogenizer and isolated by ultracentrifugation. Dilutions of test compounds were mixed with the radiotracer and membrane fraction prior to incubation for 1 h at 37° C. After a washing procedure the membranes were harvested on a filter using Skatron micro harvester. The filter spots were finally excised and counted in a Packard γ-counter. The Kd of [¹¹⁵I]-Echistatin was calculated using Prism software. Cold Echistatin was used as a reference standard and the Ki was measured to 0.14 nM. The compounds tested were EMD527040 (benchmark), the cPn216-chelated compound of Example 2(viii) and the tetraamine-chelated compound of Example 3(ii). All three compounds displayed the same behaviour (FIG. 1). The highest concentration tested was 144 μM, but even at this concentration it was not possible to compete out binding of the radiolabelled Echistatin completely.

The experiments demonstrate low affinity for αᵥβ₃ and reveal similar binding curves of the chelated versions compared to the benchmark compound EMD527040.

### Example 9

### Synthesis of Imaging Agent 7

The reaction was carried out in a manual nitrogen bubbler apparatus. The resin described in Example 4(i) (0.08 mmol) was treated with 20% piperidine in N-methylpyrrolidone for standard removal of Fmoc groups. A solution of Cy5** (prepared according to the method described in WO2008139206), (40 mg, 0.080 mmol), PyAOP (42 mg, 0.080 mmol) and diisopropylethylamine (40 μl, 0.24 mmol) in N-methylpyrrolidone (2 mL) was preactivated for 5 min and added to the resin. After 2 h HPLC analysis showed only partial conversion of the starting material and the coupling was repeated (20 h reaction time). Simultaneous removal of the Boc protecting group and cleavage of the material from the resin was performed using a mixture of trifluoroacetic acid (3 mL), dichloromethane (3 mL) and triisopropyl silane (150 μL). After 2.5 h the solution was filtered and concentrated (rotavapor). The residue was subjected to purification by preparative HPLC (column Phenomenex Luna C18(2) 5 μm 21.2×250 mm, solvents: A=water/0.1% trifluoroacetic acid and B=acetonitrile/0.1% trifluoroacetic acid; gradient 10-50% B over 60 min; flow 10.0 ml/min, UV detection at 214 nm) affording 5 mg of imaging agent 7 (1-(Cy5**-amido)-45-(3,5-dichlorophenyl)-39,43-dioxo-42-(5-(pyridin-2-ylamino)pentanamido)-3,6,9,12,15,18,21,24,27,30,33,36-dodecaoxa-40,44-diazaheptatetracontan-47-oic acid). LC-MS (column Phenomenex Luna C18(2) 20×2 mm, 3 μm, solvents: A=water/0.1% trifluoroacetic acid and B=acetonitrile/0.1% trifluoroacetic acid; gradient 10-40% B over 5 min; flow rate 0.6 mL/min, UV detection at 214 and 254 nm, ESI-MS) t_{R}=3.1 min, m/z expected 982.4 (MH)²⁺, found 982.8.

### Example 10

### Flow Cytometry Evaluation of Imaging Agent 7

Binding of imaging agent 7 (prepared as described in Example 9) was studied using flow cytometric analysis (Beckman Coulter FC500 MPL) on lung adenocarcinoma cell line H2009 (Cancer Res; 67: 5889-5895). At a concentration of 100 nM (incubation time 1 h at 37° C.) positive uptake was demonstrated in around 40% of the cells, showing that introduction of a substituent at this position in the molecule was tolerated.

What is claimed is:

1) An in vivo imaging agent of Formula I: 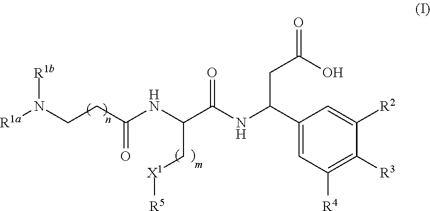
or a salt or solvate thereof, wherein:
n is an integer from 0-6;
m is an integer from 0-8;
X¹ is O, S, or NR′ wherein R′ is hydrogen or C₁₋₄ alkyl;
R¹ is —C(═NH)—NHR′ wherein R′ is as defined for X¹, or R¹ is a C₃₋₅ nitrogen-containing heteroaryl ring having 1 or 2 nitrogen heteroatoms;
each of R²-R⁴ is independently hydrogen, C₁₋₄ alkyl or halogen, or is a substituent comprising an in vivo imaging moiety, or alternatively R² and R³, or R³ and R⁴, together with the carbon atoms to which they are attached, form a C₅₋₆ aryl or C₃₋₅ heteroaryl ring having 1 or 2 heteroatoms;
R⁵ is hydrogen, or is the group R⁶R⁷ wherein R⁶ is a bivalent linker group having 1-50 bivalent linker units selected from an amino acid residue, a carbohydrate residue, —C(OH)—, —(CR′₂)—, —C(═O)—(CR′₂)—, —C(═O)—NR′—, —(CR′₂—O—CR′₂)—, —CR′₂—NR′—, CR′₂—S(O₂)—CR′₂, —(CR′₂)—O—N═CR′—, wherein R′ is as defined for X¹, and wherein R⁷ is hydrogen or is a substituent comprising an in vivo imaging moiety; and,
wherein at least one of R²-R⁴ is a substituent comprising an in vivo imaging moiety, or R⁵ is R⁶R⁷ wherein R⁷ is a substituent comprising an in vivo imaging moiety.

2) The in vivo imaging agent as defined in claim 1 wherein said in vivo imaging moiety is selected from:
(i) a radioactive metal ion;
(ii) a paramagnetic metal ion;
(iii) a gamma-emitting radioactive halogen;
(iv) a positron-emitting radioactive non-metal;
(v) a reporter suitable for in vivo optical imaging.

3) The in vivo imaging agent as defined in claim 1 wherein n is from 1-4.

4) The in vivo imaging agent as defined in claim 1 wherein m is from 0-3.

5) The in vivo imaging agent as defined in claim 1 wherein X¹ is NR′.

6) The in vivo imaging agent as defined in claim 1 wherein R¹ is a 5- or 6-membered nitrogen-containing heteroaryl ring.

7) The in vivo imaging agent as defined in claim 1 wherein each of R²-R⁴ is independently hydrogen, chloro, iodo or fluoro.

8) The in vivo imaging agent as defined in claim 1 wherein one of R²-R⁴ is ¹⁸F or ¹²³I.

9) The in vivo imaging agent as defined in claim 1 wherein R⁵ is the group R⁶R⁷.

10) The in vivo imaging agent as defined in claim 9 wherein R⁶R⁷ is the group —C(═O)—R^{6a}R^{7a} wherein R^{6a} is a bivalent linker group having 1-20 bivalent linker units, wherein said bivalent linker units is selected from an amino acid residue, a carbohydrate residue, —C(OH)—, —(CR′₂)—, —C(═O)—(CR′₂)—, —C(═O)—NR′—, —(CR′₂—O—CR′₂)—, —CR′₂—NR′—, CR′₂—S(O₂)—CR′₂, —(CR′₂)—O—N═CR′—, wherein R′ is hydrogen or C₁₋₄ alkyl and wherein R^{7a} is hydrogen or is a substituent comprising an in vivo imaging moiety.

11) The in vivo imaging agent as defined in claim 10 wherein R^{7a} comprises ¹⁸F.

12) The in vivo imaging agent as defined in claim 10 wherein R^{7a} is a metal complex, said metal complex comprising either a radioactive metal ion or a paramagnetic metal ion.

13) The in vivo imaging agent of Formula I as defined in claim 1 which is a compound of Formula Ia: 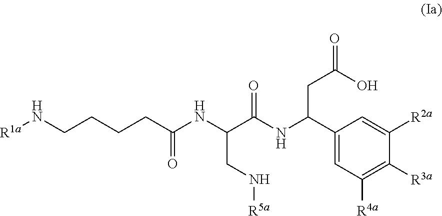
or a salt or solvate thereof, wherein:
R^{1a} is as defined for R¹ in claim 1;
R^{2a}-R^{4a} are as defined for R²-R⁴ in claim 1;
R^{5a} is hydrogen, or is the group R^{6a}R^{7a} wherein R^{6a} is a bivalent linker group having 1-50 bivalent linker units, wherein said bivalent linker units is selected from an amino acid residue, a carbohydrate residue, —C(OH)—, —(CR′₂)—, —C(═O)—(CR′₂)—, —C(═O)—NR′—, —(CR′₂—O—CR′₂)—, —CR′₂—NR′—, CR′₂—S(O₂)—CR′₂, —(CR′₂)—O—N═CR′—, wherein R′ is hydrogen or C₁₋₄ alkyl and wherein R^{7a} is hydrogen or is a substituent comprising an in vivo imaging moiety.

14) A method for the preparation of the in vivo imaging agent as defined in claim 1, wherein said method comprises reacting a suitable source of an in vivo imaging moiety with a precursor compound of Formula II: 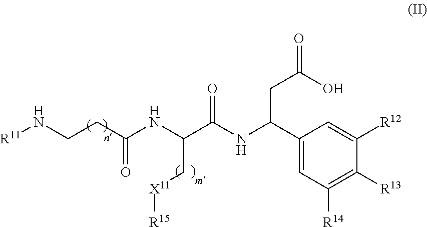
wherein:
R¹¹ is as defined for R¹ in either claim 1;
R¹²-R¹⁴ are independently selected from hydrogen, C₁₋₄ alkyl or halogen, or a precursor group;
R¹⁵ is hydrogen or a precursor group, or is the group R¹⁶R¹⁷ wherein R¹⁶ is a bivalent linker group as defined in claim 1 for R⁶, and wherein R¹⁷ is hydrogen or is a precursor group; and,
X¹¹ is as defined for X¹ in either claim 1;
m′ is as defined for m in either claim 1;
n′ is as defined for n in either claim 1;
wherein at least one of R¹²-R¹⁵ is a precursor group, or R¹⁵ is R¹⁶R¹⁷ wherein R¹⁷ is a precursor group;
and wherein any reactive groups other than said precursor group are chemically protected.

15) The method as defined in claim 14 wherein said precursor compound of Formula II is of Formula IIa: 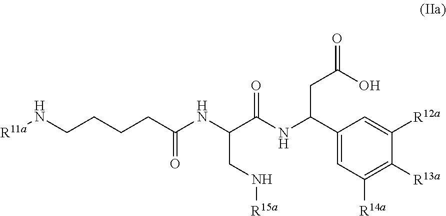
wherein R^{11a}-R^{15a} are as defined in claim 14 for R¹¹-R¹⁵ of Formula II.

16) The method as defined in claim 14 wherein said precursor group is selected from:
(i) one or more ligands capable of complexing a metallic imaging moiety;
(ii) an organometallic derivative such as a trialkylstannane or a trialkylsilane;
(iii) a derivative containing an alkyl halide, alkyl tosylate or alkyl mesylate for nucleophilic substitution;
(iv) a derivative containing an aromatic ring activated towards nucleophilic or electrophilic substitution;
(v) a derivative containing a functional group susceptible to acylation;
(vi) a derivative containing a functional group that takes part in oxime formation when reacted with a benzaldehyde;
(vii) a derivative containing a vinylsulfone functional group;
(viii) a derivative containing a functional group which undergoes facile alkylation; or,
(ix) a derivative which alkylates thiol-containing compounds to give a thioether-containing product.

17) The method as defined in claim 14 wherein one of R¹²-R¹⁴ is said precursor group.

18) The method as defined in claim 14 wherein R¹⁵ is R¹⁶R¹⁷ wherein R¹⁷ is said precursor group.

19) The method as defined in claim 14 which is carried out on a solid phase.

20) The method as defined in claim 14 which is automated.

21) A precursor compound of Formula II as defined in the method of claim 14, wherein said precursor group is selected from:
(i) a chelator capable of complexing a metallic imaging moiety; or,
(ii) an organometallic derivative such as a trialkylstannane or a trialkylsilane.

22) A cassette for carrying out the method as defined in claim 20 comprising:
(i) a vessel containing a precursor compound; and,
(ii) means for eluting the vessel with a suitable source of an in vivo imaging agent.

23) The cassette as defined in claim 22 further comprising (iii) an ion-exchange cartridge for removal of excess in vivo imaging agent.

24) A pharmaceutical composition comprising the in vivo imaging agent as defined in claim 1 together with a biocompatible carrier, in a form suitable for mammalian administration.

25) A kit for preparation of the pharmaceutical composition as defined in claim 24.

26) An in vivo imaging method to determine the quantity and/or location of αᵥβ₆ expressed in a subject, wherein said method comprises:
(i) administering to said subject the in vivo imaging agent as defined in claim 1;
(ii) allowing said administered in vivo imaging agent of step (i) to bind to αᵥβ₆ expressed in said subject;
(iii) detecting signals emitted by an in vivo imaging moiety comprised in said bound in vivo imaging agent of step (ii); and,
(iv) generating an image of the location and amount of said signals, wherein said signals represent the quantity and/or location of αᵥβ₆ expressed in said subject.

27) The in vivo imaging method as defined in claim 26 wherein said in vivo imaging agent is administered in step (i) as the pharmaceutical composition comprising the in vivo imaging agent together with a biocompatible carrier, in a form suitable for mammalian administration.

28) The in vivo imaging method as defined in claim 27 wherein said subject is a mammal.

29) The in vivo imaging method as defined in claim 28 wherein said subject is known or is suspected to have an αᵥβ₆ condition.

30) The in vivo imaging method as defined in claim 29 wherein said subject is known to have an αᵥβ₆ condition, and wherein said method is carried out repeatedly during the course of a treatment regimen for said subject, said treatment regimen comprising administration of a drug to combat said αᵥβ₆ condition.

31) The in vivo imaging method as defined in claim 26 which further comprises the step (v) of attributing the quantity and/or location of expressed αᵥβ₆ to a particular clinical picture.

32) (canceled)
